(19) United States
(12) Patent Application Publication
(10) Pub. No.: US 2026/0341442 A1
(43) Pub. Date: Oct. 29, 2026

(54) AUTOMATED RESOURCE ALLOCATION AND PREDICTION SYSTEM

(71) Applicant: Express Scripts Strategic Development, Inc., St. Louis, MO (US)

(72) Inventors: Yu Shen Lin, Barboursville, VA (US); Jonathan J. Lisic, Blacksburg, VA (US)

(21) Appl. No.: 19/188,373

(22) Filed: Apr. 24, 2025

(51) Int. Cl. G16H 20/00; G16H 10/60

(52) U.S. Cl. CPC G16H 20/00; G16H 10/60

(57) ABSTRACT

Apparatuses, systems, and methods relate to technology to receiving user data associated with a first user, wherein the user data includes usage data of the first user, and generate, with a first machine learning model executed by the computing system, a first prediction of whether the first user will receive a first treatment based on the user data. The technology further, if the first machine learning model predicts that the first user will receive the first treatment, generate, with a second machine learning model executed by the computing system, a second prediction of a future time frame that the first user will receive the first treatment based on the user data and the first prediction, and if the first machine learning model does not predict that the first user will receive the first treatment, avoid further processing of the user data and the first prediction.

### TECHNICAL FIELD

The present disclosure relates to an automated resource allocation and prediction system that automatically executes in a computing environment. Examples can include a process to determine future treatment progressions and execute automated resource allocation based on the determined future treatment progressions.

### BACKGROUND

Computing systems have become increasingly complex and sophisticated. Correspondingly, the workloads, reliance and trust in computing systems have increased. For example, computing systems can store and operate on different types of data and support numerous distinct technologies. The amount of data has significantly increased resulting in terabytes if not petabytes of information being automatically stored and modified. Accordingly, large volumes of data are being stored on computing systems.

### BRIEF DESCRIPTION OF THE SEVERAL VIEWS OF THE DRAWINGS

The various advantages of the embodiments of the present disclosure will become apparent to one skilled in the art by reading the following specification and appended claims, and by referencing the following drawings, in which:

FIG. 1 is an example of a prior artificial intelligence (AI) prediction process and enhanced AI prediction process according to an embodiment;

FIG. 2 is a flowchart of an example of a method of executing automated actions based on conditions, treatment sequences and timings according to an embodiment;

FIG. 3 is a flowchart of an example of a method of training machine learning models according to an embodiment;

FIG. 4 is an example of a modeling medical visit sequences according to an embodiment;

FIG. 5 is an example of a tabular output according to an embodiment;

FIG. 6 is an example of a probability of treatment graph according to an embodiment;

FIG. 7 is an example of a graph of predicted chemotherapy treatments according to an embodiment;

FIG. 8 is an example of a breast cancer prediction graph according to an embodiment;

FIG. 9 is an example of Cardiometabolic Syndrome according to an embodiment;

FIG. 10 is an example of key indicators that lead to specific pathway according to an embodiment;

FIG. 11 is a flowchart of an example of a method of remediating missing data according to an embodiment;

FIG. 12 is a block diagram of an example of a computing system according to an embodiment;

FIG. 13 is a block diagram of an example artificial intelligence that may be deployed within the enhanced prediction process of FIG. 1, according to some examples; and

FIG. 14 is a functional block diagram of an example neural network that can be used for the inference engine or other functions (e.g., engines) of an enhanced prediction process as described herein to produce a predictive model.

### DETAILED DESCRIPTION

Organizing, monitoring and understanding data in an enterprise can be laborious, error prone and sub-optimal due to several factors. The factors can include the complexity, scale, and diversity of the data, as well as organizational and technical barriers. Such data however can provide extremely power insights, including informed decision-making, competitive advantage, healthcare advancements, operational efficiency, predictive analytics, etc. Accordingly, significant resources have been committed to data analysis.

Big data refers to extremely large and complex datasets that traditional data-processing software cannot manage efficiently. In fact, big data can be described with three characteristics, including volume, velocity and variety. In terms of volume, a vast amount of data is generated every second in many enterprise systems. Furthermore, the velocity of data can be significant, meaning that data is generated and processed at increasingly rapid speeds. Moreover, the variety of data can include different types of data (e.g., structured, semi-structured, unstructured, different languages, different formats, etc.) from multiple sources, such as social media, internet-of-things (IoT) devices, and transactions. Other characteristics of big data can include veracity (data reliability) and value (usefulness).

Organizations can leverage big data analytics to extract valuable insights for decision-making, strategy and resource allocations. For example, enterprises can analyze customer behavior through past behavior (e.g., history, social media activity, website interactions, etc.) to generate personalized recommendations and action plans. Sentiment analysis can also seek to understand customer opinions and improve products/services. Predictive analytics can also facilitate diagnosing diseases earlier and personalizing treatment. For example, hospitals can analyze big data for patient care optimization and resource allocation. Moreover, pharmaceutical companies analyze vast clinical trial datasets to accelerate drug discovery.

Big data can be combined with artificial intelligence (AI) and machine learning (ML) to enable businesses and organizations to make data-driven decisions, increasing efficiency, perform intelligent resource allocations and enhancing user experiences. The continuous growth of data and advancements in analytics tools is influencing the future of various industries. Indeed, predictive analytics for early diagnosis and treatment is being extensively studied. AI and machine learning can analyze patient data (e.g., medical records, genetic information, lifestyle habits) to detect patterns and predict diseases like cancer, diabetes, or heart conditions before symptoms appear or increase in scope and magnitude. In some examples, wearable devices (e.g., smart watches, heart rate monitors, blood pressure cuffs, etc.) can monitor real-time health metrics, alerting users and doctors about irregularities and can provide valuable data.

In some cases, personalized medicine and treatment plans can be generated. For example, big data enables precision medicine by analyzing a patient's DNA, medical history, and environmental factors to tailor treatments. Moreover, AI can facilitate recommend targeted therapies instead of a “one-size-fits-all” approach. Furthermore, digital health records can integrate data from hospitals, clinics, pharmacies, and insurance companies, making information accessible across systems. Predictive analytics can facilitate hospitals procedures, optimize patient flow, reduce wait times and improve efficiency. IoT and wearable health devices track vitals (heart rate, glucose levels, oxygen saturation) and send data to doctors in real time for interventions. Accordingly, big data can enhance healthcare outcomes, reducing costs, and making medical services more efficient.

Previously existing implementations of big data relied heavily on expert rule systems and/or AI to analyze the big data. Reliance on expert rule systems can provide valuable insight but is prone to error and obsolescence. For example, in rapidly evolving fields, expert rules can become quickly obsolete (e.g., fails to match new, evolving and emerging data patterns) and generate inaccurate or sub-optimal results. Consequently, experts rule systems (which rely on expert rules) often include significant overhead to update and adjust. Moreover, such expert rules systems are also prone to delays since first new updates (e.g., new data patterns) are identified and then rules to identify the patterns and consequences are manually coded. Doing so is impossible to execute in real time, and in some cases never occurs if the new data patterns are not identified.

For example, the emergence of new diseases (e.g., Coronavirus disease (COVID-19)) rapid emergence can take years to fully understand. Consequently, generating an expert rules system to identify and treat COVID-19 can consume years limiting the effectiveness and useability of expert rules systems.

AI systems can analyze big data. AI is often sub-optimally applied and inefficiently applied in such contexts. For example, AI can be a powerful tool that generates insights in real time at a rapid pace. AI however can consume massive amounts of resources. For example, deep learning and large-scale machine learning models rely on massive computing resources. The demand varies depending on the type of AI model, complexity, and the application but is significant.

Training AI Models relies on extensive resources, vast datasets and powerful hardware. For example, neural networks process millions to billions of parameters with thousands of high-performance GPUs (Graphics Processing Units) and/or TPUs (Tensor Processing Units) that execute over months. Consequently, cloud computing is often used for training since local machines lack sufficient processing power increasing costs and latency.

Inference can be less resource intensive than training but still represents a significant demand. Once an AI model is trained, the AI model still needs computing resources to process inputs and generate outputs. Cloud-based AI systems can optimize inference efficiency with specialized hardware, but doing so is both costly and can create latency due to waiting for the specialized hardware to become available.

AI also consumes massive amounts of energy to execute. AI data centers consume enormous amounts of electricity and water, leading to concerns about energy and water availability, pricing and carbon emissions. In some cases, the AI data centers can operate at sub-optimal speeds and with reduced processing because power is unavailable.

Furthermore, numerous AI based systems are often trained and deployed. For example, each of the AI based systems can be deployed for a different purpose (e.g., each AI based system produces different outputs based on different data). Accordingly, the problems mentioned above are amplified since numerous AI models are employed.

Thus, the previous AI applications and expert rules systems suffer from several problems noted above. For example, expert rules systems are prone to error, rapid obsolescence, significant overhead, delays, failure to be updated in real time, etc. AI based systems rely on extensive resources, vast datasets, powerful hardware, cost, increased latency, massive amounts of energy and water, sub-optimal speeds, etc., which are often times amplified by the significant amount of AI models that are trained and executed.

Enhanced examples herein include a solution to address the aforementioned technical challenges with modern tools and a clear strategy to reduce the overhead of big data analysis and generate more value from big data by leveraging AI analysis in a streamlined and efficient manner. Solutions described herein receive user data associated with a first user, where the user data includes usage data of the first user, generate, with a first machine learning model, a first prediction of whether the first user will receive a first treatment based on the user data, if the first machine learning model predicts that the first user will receive the first treatment, generate, with a second machine learning model, a second prediction of a future time frame that the first user will receive the first treatment based on the user data and the first prediction, and if the first machine learning model does not predict that the first user will receive the first treatment, avoid further processing of the user data and the first prediction.

Doing so can reduce big data analysis overhead (e.g., processing power, hardware resources, etc.) and latency because only two machine learning models are trained and operated. For example, enhanced examples herein reduce the amount of AI models by a significant margin relative to existing designs. Doing so can reduce the resources for training we well as inference while also providing enhanced and accurate data analysis (e.g., treatments, time frames for the treatments and diagnoses). Indeed, examples can efficiently and accurately allocate resources and perform automated actions based on the predictions of the AI models.

Accordingly, the claimed solution is necessarily rooted in computer technology (AI technology) in order to overcome the aforementioned technical problems specifically arising in the realm of computer environments. That is, examples are an improvement to computer technology and are firmly rooted in computer technology through the generation and implementation of enhanced AI models.

Doing so for a human being can be difficult if not impossible to do. As noted above, terabytes or petabytes of information can exist in an enterprise system. Accordingly, identifying, retrieving and linking data to generate an accurate prediction of future actions and/or treatments for a user can be impossible for a human being to manually execute consistently, accurately and in real-time. For example, human beings cannot identify the complex patterns that lead to a series of actions, treatments and time frames particularly as data pattern identification in certain contexts (e.g., medical environments) can be overwhelmingly complex and is often-time inaccurate. For example, on average, annually, around fourteen million customers utilize 600 million medical claims. For oncology, about 1.7% of the fourteen million customers had a true diagnosis of cancer and require treatment (e.g., surgery, chemotherapy and/or radiation therapy). Examples (e.g., an Enhancing Oncology Model (EOM) and/or visit sequence model (VSM)-oncology model comprising the first and second machine learning models) herein were able to identify such cancerous patients before the treatment started and/or diagnosis. For cardiometabolic syndrome (CMS), about 1.4% of the 14 million customers would have a cancerous disease progress to stage 3 and 1.5% would progress to stage 4.

Similarly, examples can also include a VSM that is applied to M usculoskeletal (MSK) diseases (a VSM-MSK model). The VSM-MSK model was able to identify MSK patients before disease deterioration and progression. For MSK diseases, a significant percentage of afflicted customers of the 14 million customers had surgery to treat the MSK diseases. The VSM-MSK model was able to identify such users prior to surgery potentially resulting in other interventions (e.g., medications, physical therapies, etc.) instead surgeries resulting in significant savings and high quality outcomes for the customers.

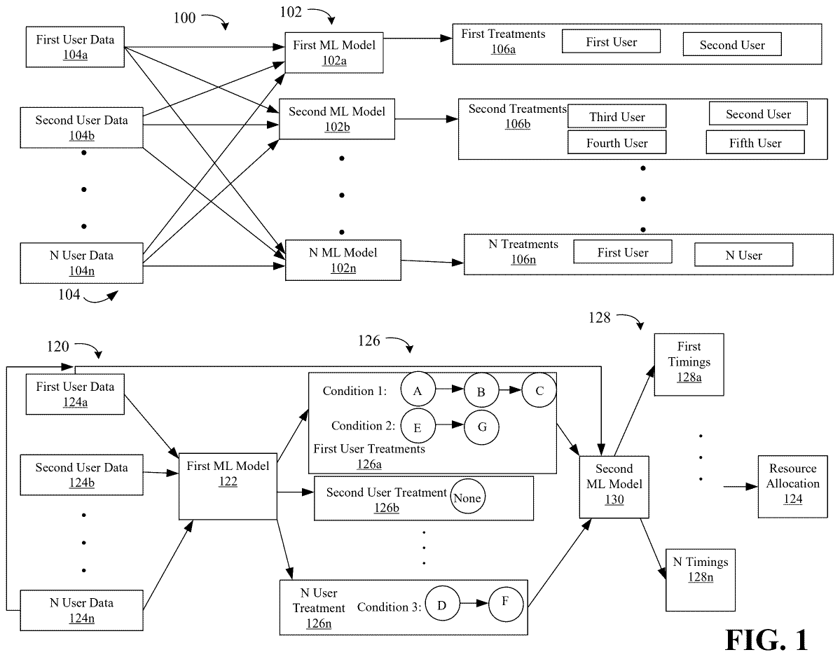

Turning now to FIG. 1, a prior AI prediction process 100 is provided. While the prior AI prediction process 100 and enhanced prediction process 120 are discussed below, it will be understood that the details described herein can apply to other contexts and applications as well.

The prior AI prediction process 100 can generate different predicted treatments (e.g., medical treatments such as doctor visits, chemotherapy, cardiometabolic visits, etc.) for different users. The prior AI prediction process 100 relies on user data 104 including first user data 104a, second user data 104b-N user data 104n (some user data is not illustrated for brevity). The first user data 104a-N user data 104n can correspond to different users and include any number of distinct data. For example, the first user data 104a can correspond to a first user (e.g., include health data of a first user), second user data 104b can correspond to a second user (e.g., include health data of a second user), third user data can correspond to a third user (e.g., include health data of the third user; not illustrated), N user data 104n can correspond to an N user (e.g., include health data of an N user) and so on.

The first user data 104a-N user data 104n is provided to ML models 102. The ML models 102 can process the first user data 104a-N user data 104n to generate different predictions. Notably, each of the ML models 102 can determine a different treatment. For example, a first ML model 102a can generate a first prediction that includes a first treatments 106a (e.g., liver cancer treatments for liver cancer patients) for the first-N users. That is, the first ML model 102a is trained to predict a treatment course for liver cancer patients. Notably, the first ML model 102a is unable to predict treatments for other diseases and/or any other diseases beyond liver cancer.

A second ML model 102b can generate a second prediction that includes a second treatments 106b (e.g., diabetes treatments for diabetic patients) for the first-N users. That is, the first ML model 102a is trained to predict a treatment course for diabetic patients. Notably, the second ML model 102b is unable to predict treatments for other diseases and/or any other diseases beyond diabetes.

An NML model 102n can generate an N prediction that includes an N treatments 106n (e.g., arthritis treatments for arthritic patients) for the first-N users. That is, the NML model 102n is trained to predict a treatment course for arthritic patients. Notably, the NML model 102n is unable to predict treatments for other diseases and/or any other diseases beyond arthritis.

Accordingly, the first ML model 102a generates first treatments 106a for the first user and the second user (liver cancer patients). The first treatments 106a can include a single predicted treatment for the first user to treat the liver cancer of the first user, and a single predicted treatment for a second user and to treat the liver cancer of the second user. The predicted treatment for the first user can be different than the predicted treatment for the second user.

Similarly, the second ML model 102b generates second treatments 106b (e.g., diabetes treatments for diabetic patients) for the first user, second user, third user (user data not specifically illustrated but is provided to the second ML model 102b) and fifth user (user data not specifically illustrated but is provided to the second ML model 102b). Accordingly, the second treatments 106b can include different single treatments for each of the second, third, fourth and fifth user, where the single treatments can be different from each other.

Likewise, the NML model 102n generates N treatments 106n for the first user and N user (e.g., arthritis treatments for arthritic patients). Accordingly, the N treatments 106n can include single treatments for each of the first and N users, where the single treatments can be different from each other. Other models (not illustrated) can generate various treatments as well.

Notably, the first user data 104a-N user data 104n is provided to each of the ML models 102. Doing so increases bandwidth and communication overhead since data is repeatedly and inefficiently transmitted from servers and/or data warehouses that store the first user data 104a-N user data 104n to AI data centers that execute the ML models 102.

Moreover, having the numerous ML models 102 significantly increases consumed energy, hardware overhead (e.g., GPU and/or TPUs usage is increased), memory overhead and latency. For example, the first user data 104a-N user data 104n is replicated multiple times to be provided to each of the ML models 102 increasing memory usage to be stored for each of the ML models 102. Moreover, having such a large number of ML models 102 utilizes a significant amount of resources as noted above suffering from the problems stated above with respect to the previous AI applications.

The ML models 102 are predictive models in healthcare that focus on single treatment and lack the ability to determine the timing or stage of the treatment. That is, the ML models 102 are unable to determine a sequence of treatments and instead determine a single future treatment. This gap in the ML models 102 can hinder the ability to provide appropriate and timely care to patients at different stages of the treatment journey of the patients. That is, the prior AI prediction process 100 and in particular the ML models 102 are unable to predict time frames for the first treatments 106a-N treatments 106n, thus failing to provide adequate decision-making information.

Notably as well, the diagnoses and the treatments for the first-N users are generated by different models of the ML models 102. For example, the first user has treatments listed in the first treatments 106a and treatments listed in the N treatments 106n. Such distinct groupings of treatments that apply to a same patient can result in confusion and poor prioritization of the treatments. For example, a proper priority would indicate that the first user is treated for liver cancer as soon as possible and that the arthritis treatments can be treated at a later time. Due to misunderstanding and/or miscomprehension, the opposite can occur (arthritis treated before liver cancer) and resources (both computer and human) are allocated to the arthritis treatment prior to the liver cancer. That is, the prior AI prediction process 100 fails to build a full understanding and/or comprehension of all of the different treatments and diagnosis for a particular user resulting in confusion and sub-optimal progressions.

Turning now to enhanced prediction process 120, first user data 124a-N user data 124n is provided. Similar to above, each of the first user data 124a-N user data 124n corresponds to a different user, meaning that the first user data 124a corresponds to a first user, second user data 124b corresponds to a second user, and the N user data corresponds to an N user. Unillustrated user data (e.g., a third user data) are also provided. In this example, each of the first user data 124a-N user data 124n is provided to a first ML model 122 (e.g., a single model). The first ML model 122 is trained to determine various conditions and treatments. The first ML model 122 and a second ML model 130 (which indicate the incident/care plan, and days to events respectively) are agnostic to model choices, provided that the first ML model 122 and second ML model 130 provide a classification for incident and a non-negative continuous response for days-to-event. Examples of the first ML model 122 and a second ML model 130 include gradient-boosted trees (XG Boost, LightGBM, Random Forest, Linear Regression etc.).

The first user data 124a-N user data 124n can include various data relating to the first-N users. For example, the first user data 124a can include real-time health metrics (resting heart rate, heart rate variability, breathing rate, skin temperature and oxygen saturation, etc.) of the first user that are received from an electronic device (e.g., from a wearable device such as a heart rate monitor, smartwatch, etc.) of the first user that are uploaded and stored. The first user data 124a can also include records (e.g., electronic health records, claim records such as medical insurance claims, previous treatment records, doctor notes, etc.) that reflect the first user's historical medical diagnoses and treatments. The second user data 124b likewise can include similar information relating to the second user, the N user data 124n can include similar information relating to the N user and so forth.

Available types of data that were considered to form part of the first user data 124a-N user data 124n can include customer information such as demographics information (e.g., age, gender, race, social determinants of health (SDI), zip, industry (job type), income, education, biometric information (e.g., body mass index (BMI), Low-Density Lipoprotein (LDL), blood sugar, A1c test results, blood pressure (BP) results), lifestyle information (e.g., smoking, weight, exercise, alcohol, fatty food, high fiber food, sleep, etc.), and enrollment (e.g., type of insurance, how long has user been insured, coverage, etc.). The available types of data that were considered included medical claim information such as diagnosis (dx1-dx28), medications (e.g., blood pressure medications, LDL medications, depression medications, dosage medications, opioid usage), services received (e.g., office visits, radiology, advanced imaging, lab, rehab etc.), location of services (e.g., inpatient services (IP), emergency room (ER), urgent care, etc.), provider specialty, utilized pharmacy information (e.g., cost, drug class, days supply, etc.), pain management visits such as injection, no-injection (e.g., physical therapy (PT)), behavior cognitive therapy, etc. The available types of data that were considered also include providers requests for authorization and/or pre-authorization such as advanced imaging (Magnetic resonance imaging (MRI), computed tomography (CT) scans), surgery, chemotherapy, radiation therapy, admissions etc. The available types of data that were considered included providers information such as demographics, affiliation, contract type (e.g., fee for service, case rate, health maintenance organization), size of office (e.g., number of providers).

In some examples, a subset of relevant data can be used as part of the first user data 124a-N user data 124n and the remaining types of data are omitted and/or ignored. Exemplary examples of the subset are as follows and can include customer information including demographics information for a respective user including age, gender, race, SDI, zip, industry (job type of the user), income, education; biometric information (BMI, LDL, A1c test results, BP results), and lifestyle information (smoking, weight, exercise, alcohol, fatty food, high fiber food and sleep patterns). The subset can also include past utilization such as Primary Care Provider (PCP)/specialist care provider (SPC) visit, lab testing, radiology (e.g., X-ray, MRI/CT), IP visits, ER visits, pain management, and prescription utilization. The subset can also include claim information such as diagnosis (dx1-dx28), services (e.g., Office Visits, Radiology, Advanced Imaging, Lab, Rehab, etc.), location of services (IP, ER, Urgent Care etc.), provider specialty, utilized pharmacy information (e.g., cost, drug class, days supply). The subset can also include providers requests for authorization and/or pre-authorization such as advanced imaging (M RI, CT, etc.), surgery, chemotherapy, radiation therapy, admission, etc. The subset can also include medications (e.g., blood pressure medications, LDL medications, depression medications, dosage medications, opioid usage).

In some examples, particularly salient data types from the subset are focused on in some examples while omitting other data from consideration. For example, the salient data types can include demographics information for a respective user including age, gender, race, SDI, job type, education, income, etc. The salient data types include past utilization such as Primary Care Provider (PCP) /specialist care provider (SPC) visit, lab testing, radiology (e.g., X-ray, M RI/CT), IP visits, ER visits, pain management visits, and prescription utilization. The salient data types further include biometric information such as A1c, LDL, BMI and BP results. The salient data types include medications (e.g., blood pressure medications, LDL medications, depression medications, dosage medications, opioid usage). The salient data types can include lifestyle such as exercise, smoking, alcohol, fatty food, high fiber food and sleep patterns. The salient data types can include pain management such as injection, no-injection (e.g., i.e., PT), behavior cognitive therapy, etc.

The first ML model 122 can determine first user treatments 126a based on the first user data 124a. For example, the first user treatments 126a can be determined based on patterns (e.g., previous treatments and diagnoses) in the first user data 124a. The first ML model 122 can categorize the first user into a first group (e.g., has liver cancer) from a plurality of groups based on the first user data 124a, and generate first user treatments 126a (e.g., the first prediction, and specifically treatment sequence A, B, C) based on the first group. The first ML model 122 can categorize the first user into a second group as well (e.g., has high blood pressure) from the plurality of groups based on the first user data 124a, and generate first user treatments 126a (e.g., the first prediction and specifically the treatment sequence E, G) based on the second group.

The first user treatments 126a can be specific to the first user. In this example, the first ML model 122 determines that the first user has two different conditions shown as condition 1 and condition 2 (e.g., liver cancer, arthritis, diabetes, mental health, etc.). The first ML model 122 further determines that condition 1 has a treatment course of A, B and C (A leads to B and B leads to C). That is, A occurs first, B occurs second and C occurs third. Accordingly, a sequence of treatments A, B, C (e.g., cancer treatments such as chemotherapy A, B followed by surgery C) are predicted to occur to treat condition 1. Similarly, the first ML model 122 determines that condition 2 has a treatment course of E and G where E leads to G (e.g., COVID-19 treatments as such as medication administration show as “E” and follow up-visit shown as “G”). Accordingly, a sequence of treatments E, G are predicted to occur to treat condition 2. Accordingly, the conditions 1 and 2 and corresponding treatments can be generated and stored together (e.g., in a same data structure). That is, all of the conditions 1, 2 of the first user are readily apparent in a single consolidated location to provide an accurate overview of the first user to providers or other parties (e.g., doctors, insurance company, therapist, etc.) that are involved in the treatment of conditions 1, 2. Doing so can enable a snapshot of the first user and quickly facilitate different treatments.

Further, the first ML model 122 generates both of the sequence of treatments A, B, C to treat condition 1, and the sequence of treatments E, G to treat condition 2. Doing so has several enhancements, including avoiding or reducing interactions between the sequence of treatments A, B, C and sequence of treatments E, G. For example, if the treatments for condition 1 and 2 were separately generated by different ML models (such as first ML model 102a and second ML model 102b in the prior AI prediction process 100) there is potential for complications since the different ML models are unaware of the other treatments. The first ML model 122 generates and is aware of both the sequence of treatments A, B, C for condition 1 and sequence of treatments E, G for treatment 2, which reduces complications since the first ML model 122 can avoid using treatments known to cause complications with each other selected treatments.

Similarly, the first ML model 122 can output second user treatments 126b based on second user data 124b. That is, the second user treatments 126b can only apply to the second user. In this example, the second user has no conditions (e.g., medical conditions) and accordingly no treatments are listed.

Furthermore, the first ML model 122 generates an N user treatment 126n based on the N user data 124b. The N user treatment 126n can be applicable to the N user. In this example, the N user has condition three with a treatment progression of treatment D to treatment F. Accordingly, a sequence of treatments D, F are predicted to occur to treat condition 3. The first ML model 122 can generate other treatment conditions (unillustrated) for different users.

Accordingly, the first ML model 122 can generate treatments for the first-N users. A subset of the treatments from the first-N user treatments 126a-126n are provided to a second ML model 130 (a single model). That is, the first-N user treatments 126a-126n are filtered based on which of the first-N user treatments 126a-126n include a diagnosis and treatment progression. For example, the second user treatments 126b would be filtered and removed since no treatment progression or diagnosis was found for the second user. Meanwhile, the first user treatments 126a and the N user treatment 126n would not be filtered (would be maintained) based on the first user treatments 126a and the N user treatment 126n including diagnoses and treatment sequences. The subset is provided to the second ML model 130, where the subset includes the first user treatments 126a and the N user treatment 126n among any other user treatments from the user treatments 126 that include diagnoses and treatment.

Examples can likewise filter the first user data 124a-N user data 124n. For example, data associated with no diagnosis and treatment is removed. In this case, the second user data 124b is filtered and not provided to the second ML model 130 since the second user treatments 126b, which is generated based on the second user data 124b, indicates that no user treatment or diagnosis. Accordingly, the second ML model 130 can receive the first user data 124a and N user data 124n.

The filtering of the first user data 124a-N user data 124n and the first-N user treatments 126a-126n described above can reduce bandwidth since less data is transmitted to the second ML model 130. Furthermore, processing power and speed are improved since the second ML model 130 operates on less data.

The second ML model 130 can generate future time frames for the treatment progressions. For example, first timings 128a can be generated based on the first user treatments 126a and the first user data 124a. The first timings 128a can indicate when the treatment progression A, B, C will occur (e.g., treatment A will occur in 7 days from a first date of diagnosis of condition 1 in the first user, treatment B will occur in 14 days from the first date, treatment C will occur in 21 days from the first date). The first ML model 122 can predict when treatment progression E, G will occur (e.g., treatment E will occur in 60 days from a second date of diagnosis of condition 2 in the first user, treatment G will occur in 90 days from the second date).

Likewise, the N timings 128n can include windows of time (e.g., days) when treatments D and F will occur. Other timings can be generated, but notably timings are not generated for the second user treatment 126b since no treatment progression and diagnosis is identified.

The timings 128 and the subset of user conditions can guide resource allocation 124. For example, electronic outreach (e.g., automated email, text message, etc.) can be generated, scheduled and/or executed to inform users of the expected treatment progression, timings 128 and diagnoses. Doing so can conserve computing resources by avoiding mass communications (e.g., emails, texts, etc.) that are often overlooked by users. In contrast, examples herein can craft focused and relevant electronic communications to discuss the specific treatments and diagnoses of the first-N users. For example, information explaining condition 1 and 2, expected outcomes, treatment sequence A, B, C, treatment sequence E, G, timings 128 etc. can be communicated to the first user.

For example, another resource allocation case is as follows. The cost for a case manager at around $50 per case while a self-serve digital coaching (e.g., potentially machine learning and/or artificial intelligence related) is significantly lower at about $1 per case. The first ML model 122 and second ML model 130 can triage customers to case management via the case manager, and/or to the self-serve digital coaching.

For example, suppose that two cases have CMS. In a first case, the first user experiences an emergency room visit, a blood culture and antibodies medication as indicated in first user data 124a. The first ML model 122 can predict based on the first user data 124a that a high risk for stage 3 infection exists, and the first user treatments 126a can include infection prevention for the infection. In this first case, first user treatments 126a can further include an allocation of a case manager's resources to help the first user align with treatment to prevent progress to stage 3.

In a second case as represented in second user data 124b, suppose that the second user had an office visit and a simple lab test with no medication and no follow up visit. The first ML model 122 would predict that the second user is at a stable stage 1 for infection. The first ML model 122 would then assign the second user for digital coaching, reducing resources (e.g., a savings of $49 relative to a case manager assignment), time and latency to treat the second user. Further, as noted above, the first ML model 122 does not assign a digital coaching for the first user to conserve computing resources saving computing resources. When considered in view of the enormous amount (e.g., three-million or more) CMS cases, in stage 1 and/or ? stage2, the savings and enhancements are significant.

In some examples, an appointment can be automatically scheduled with a medical professional to explain the diagnosis (condition 1 and 2), and treatment sequences A, B, C and E, G. Providing the first user with such information can improve the overall outcome of the treatments A, B, C, E, G since the first user understands the progression and optimal timings, and is more likely to adhere to the treatment schedule.

In some examples, an appointment to perform a treatment can be automatically scheduled. For example, treatment A for the first user can be automatically scheduled to occur at 7 days from the first date of diagnosis of condition 1 in the first user based on electronic calendars of providers. For example, the electronic calendars of providers with the capacity (e.g., expertise) to perform treatment A can be accessed. A subset of the providers that have availability (e.g., vacancies) during the window of time can be selected. A nearest provider from the subset of the providers to the first user (and/or highest ranked provider) can be selected and an appointment to schedule treatment A is scheduled with the nearest provider. In some examples, a combination of distance and/or provider rank is used to select the provider.

In some examples the most exigent of the diagnosis (e.g., condition 1 or condition 2) can be identified and prioritized. For example, if condition 1 is liver cancer and condition 2 is high blood pressure, examples can prioritize the treatment sequence A, B, C, for condition 1 rather than condition 2 by allocating computing resources (e.g., electronic messages, automated scheduling, etc.) to ensure that treatment sequence A occurs in a timely fashion and according to the first timings 128a.

In some examples, the first ML model 122 generates each condition to have a probability of occurrence. Certain conditions that have probabilities below a threshold are discarded. Further, these examples are readily combinable. For example, the enhanced prediction process 120 can determine, with the first machine learning model 122, a plurality of predictions (e.g., first user treatments 126a) based on the first user data 124a, where the plurality of predictions include different treatments (e.g., treatment sequence A, B, C and treatment sequence E, G) of the first user that are associated with different probabilities and conditions (e.g., condition 1 and 2 have different probabilities) of the first user, group the plurality of predictions into different groups (e.g., exigent group or not exigent group) and select a first prediction (e.g., treatment sequence A, B, C) from the plurality of predictions based on the first prediction having a probability from the different probabilities that is above a threshold and being grouped into a high-risk group (e.g., exigent group) from the different groups.

Accordingly, resources are more efficiently allocated to users that have the most pressing conditions and treatments. That is, examples prioritize the treatments that will occur soonest in time and allocate digital and human resources accordingly. Notably, examples can include only two machine learning models including the first ML model 122 and the second ML model 130. Relative to prior AI prediction process 100, the enhanced prediction process 120 operates with reduced latency, bandwidth processing power, memory and resources as only the first ML model 122 and the second ML model 130 are executed as opposed to the numerous models ML models 102 (e.g., each diagnosis is analyzed by a different ML model of the ML models 102). Moreover, the new functionality is provided as the first timings 128a-N timings 128n are determined which the prior AI prediction process 100 was unable to determine.

As shown in prior AI prediction process 100, traditionally predictive models in healthcare often focus on a single treatment per model, and lack the ability to determine the timing or stage of the treatment. This gap in the AI models 102 can hinder the ability to provide appropriate and timely care to patients at different stages of their treatment journey. As shown in enhanced prediction process 120, enhanced examples herein predict a sequence and timing of treatments for individual users. Numerous parties can access the timings 128 and the user treatments 126, including case managers, coaches, care solution teams, care coordination teams, etc. to implement as much.

As one specific example, consider oncology care. The enhanced prediction process 120 can predict both a next treatment and timing for the next treatment. For example, the first ML model 122 and the second ML model 130 can predict that Patient A will undergo surgery within the next 30 days, followed by chemotherapy 14 days later without radiation therapy. Meanwhile, the first ML model 122 and second ML model 130 can predict that Patient B might will receive chemotherapy within the next 7 days followed by another round of chemotherapy 14 days later, and then radiation therapy 30 days later. In contrast, the first ML model 122 and second ML model 130 can predict that Patient C has no treatment planned for the next 180 days. Such a powerful and granular approach provides clinicians with a clear understanding of each patient's treatment stage, enabling hospitals, clinics, electronic processes, etc. to offer tailored care and communication based on individualized treatments and timings. Doing so provides significant enhancements, particularly because humans and prior art systems are unable to accurately consistently assess cancer diagnoses. For example, annually, about 2.4 million users had suspicious cancer diagnosis. Only around 10% of the 2.4 million users were finally diagnosed as having cancer. The first ML model 122 and the second ML model 130 provide an accurate and consistent way to access cancer diagnoses that enables high-precision focus on the 240,000 customers instead of all 2.4 million that is unable to be performed by a human.

The first user data 124a-N user data 124n can be medical data, for example medical claims data. The medical claims data can be categorized based on user. For example, the first user data 124a can include medical claim data of the first user, second user data 124b can include claim data of the second user etc. Leveraging claim data to identify certain patients (e.g., cancer patients can be challenging due to a high rate of false positives, where a significant portion of patients with suspicious symptoms do not actually having cancer. This situation often leads to the allocation of substantial clinical and computing resource for triage. The enhanced prediction process 120 has the capability to accurately predict which patients truly need to undergo cancer treatment and can be immensely valuable. By distinguishing between patients who should receive treatment and patients who should not receive treatment, examples can facilitate clinical operation team's allocation of resources more efficiently. Doing so can lead to substantial resource savings and streamlined clinical operations.

Consider the case of a patient with stage 4 lung cancer in a rural community. In this scenario the community oncologist may not have access to the information regarding new treatments, further impacting health outcomes. A cancer treatment sequence and timing for the patient can be generated with the first ML model 122 and the second ML model 130, to estimate the trajectory of the patient. The resource allocation 124 can be adjusted based on the cancer treatment sequence and timing allowing examples to electronically connect the community oncologist with a seasoned and knowledgeable oncologist consultation service who can inform the community oncologist of new and advisable treatments. Not only does the above impact the outcome of this single patient, but through sharing this expertise and knowledge, examples can help bridge knowledge gaps and ensure that all patients receive the most up-to-date and effective care. Accordingly, some examples can connect third-party providers based on the outputs of the first ML model 122 and the second ML model 130.

Furthermore, the first ML model 122 and second ML model 130 can be applied to any type of condition, including physical and mental (e.g., substance use disorders). Thus, examples can use the first ML model 122 to generate a sequence of treatments (e.g., therapy sessions, rehabilitation chemotherapy, etc.) for numerous conditions, and the second ML model 130 to generate timings for the sequences of treatments.

In some examples, enhanced prediction process 120 can identify total medical data associated with the first-N users and filter the total medical data to generate the first user data 124a-N user data 124n. For example, enhanced prediction process 120 can determine a window of time (e.g., five years) based on a history of the first user (e.g., first user has cancer history in the family) and filter the total medical data (e.g., medical data that spans 20 years) based on the window of time to generate the first user data 124a. Thus, the history can relate to a specific concern, medical issue, or family history. The window of time can be used to remove aged data that is no longer relevant.

One or more of the features above can be connected via networks. For example, the first user data 124a-N user data 124n can be transmitted to the first ML model 122 and the second ML model 130 through networks. Additionally, the first ML model 122 and the second ML model 130 can be connected through a network. connected to each through networks. The network(s) connecting the various components of enhanced prediction process 120 can include, or operate in conjunction with, an ad hoc network, an intranet, an extranet, a virtual private network (VPN), a local area network (LAN), a wireless network, a low energy Bluetooth (BLE) connection, a WiFi direct connection, a wireless LAN (WLAN), a wide area network (WAN), a wireless WAN (WWAN), a metropolitan area network (MAN), the Internet, a portion of the Internet, a portion of the Public Switched Telephone Network (PSTN), a plain old telephone service (POTS) network, a cellular telephone network, a wireless network, a Wi-Fi® network, another type of network, or a combination of two or more such networks. For example, a network or a portion of a network can include a wireless or cellular network and the coupling can be a Code Division Multiple Access (CDMA) connection, a Global System for Mobile communications (GSM) connection, or other type of cellular or wireless coupling. In this example, the coupling can implement any of a variety of types of data transfer technology, such as Single Carrier Radio Transmission Technology (1xRTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, fifth generation wireless (5G) networks, Universal Mobile Telecommunications System (UMTS), High Speed Packet Access (HSPA), Worldwide Interoperability for Microwave Access (WiMAX), Long Term Evolution (LTE) standard, others defined by various standard setting organizations, other long range protocols, or other data transfer technology.

Any and all aspects of the enhanced prediction process 120 can be implemented be implemented in logic instructions (e.g., software), configurable logic, fixed-functionality hardware logic, computer readable instructions stored on at least one non-transitory computer readable storage medium that are executable to implement aspects of the enhanced prediction process 120, circuitry, etc., or any combination thereof. The enhanced prediction process 120 can be a computing architecture, in which any of the components are executed in logic instructions (e.g., software), configurable logic, fixed-functionality hardware logic, computer readable instructions stored on at least one non-transitory computer readable storage medium that are executable to implement on the enhanced prediction process 120, circuitry, etc., or any combination thereof.

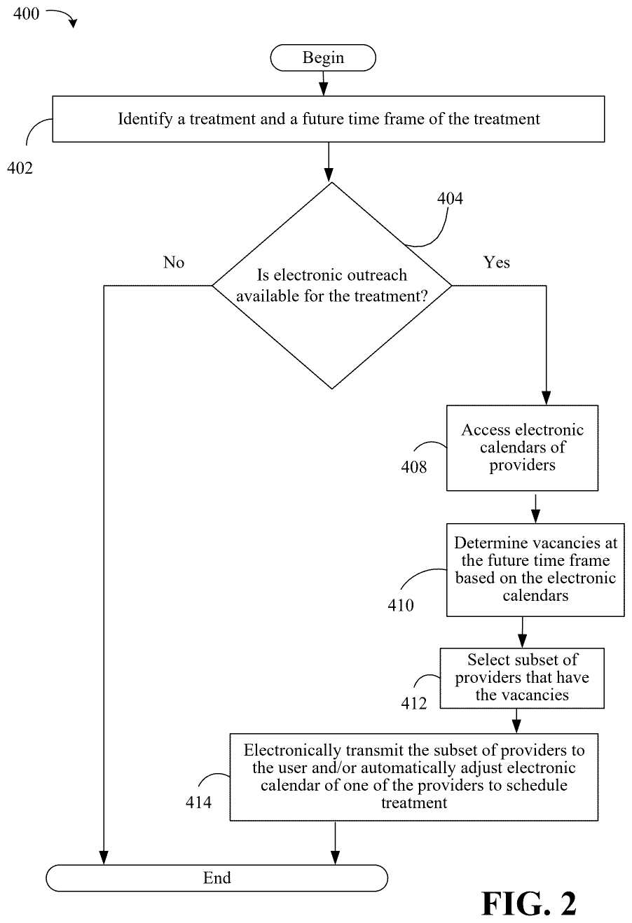

Turning now to FIG. 2, a method 400 for executing automated actions based on conditions, treatment sequences and timings is illustrated. The method 400 can be readily implemented in the example of the enhanced prediction process 120 (FIG. 1). In an embodiment, method 400 is implemented in logic instructions (e.g., software), configurable logic, fixed-functionality hardware logic, computer readable instructions stored on at least one non-transitory computer readable storage medium that are executable to implement method 400, circuitry, etc., or any combination thereof.

Illustrated processing block 402 can identify a treatment (e.g., generated with the first ML model 122 of FIG. 1) and a future time frame (e.g., generated with first ML model 122 of FIG. 1) of the treatment. As noted above, the treatment can include a treatment progression of a series of actions.

Illustrated processing block 404 determines if electronic outreach is available for the treatment. If so, illustrated processing block 408 accesses electronic calendars of providers that can execute the treatment. Illustrated processing block 410 determines vacancies at the future time frame based on the electronic calendars. For example, if an electronic calendar of a provider shows a vacancy within the future time frame, the provider has availability. If, however, the electronic calendar shows no vacancies at the future time frame, the provider shows does not have vacancies within the future time frame.

Illustrated processing block 412 selects a subset of providers that have vacancies. For example, processing block 412 can identify the subset based on a number of factors, including distance from the user, specialties of the providers, whether the providers can support the treatments, etc. Illustrated processing block 414 electronically transmits the subset of the providers to the user and/or automatically adjusts the electronic calendar of the one of the providers to schedule treatment. If the electronic outreach is unavailable at processing block 404, examples can cease processing. In some examples, the electronic outreach can include providing notifications to the user and/or information to the user.

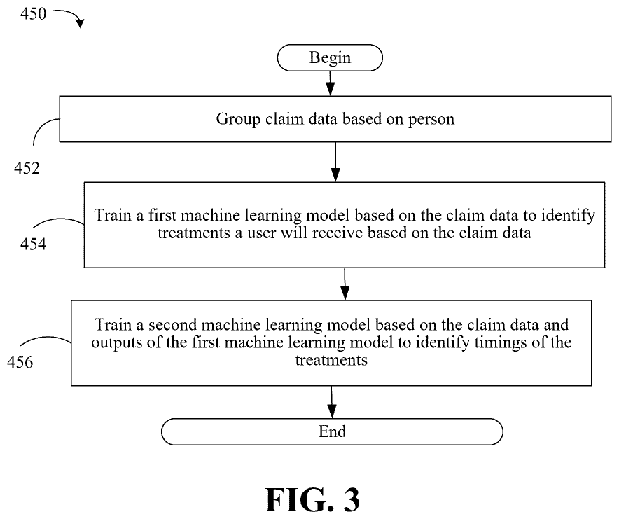

Turning now to FIG. 3, a method 450 for training machine learning models is illustrated. The method 450 can be readily implemented in the example of the enhanced prediction process 120 (FIG. 1) and/or method 400 (FIG. 2). In an embodiment, the method 450 is implemented in logic instructions (e.g., software), configurable logic, fixed-functionality hardware logic, computer readable instructions stored on at least one non-transitory computer readable storage medium that are executable to implement method 400, circuitry, etc., or any combination thereof.

Illustrated processing block 452 groups claim data based on person. For example, the claim data can be structured data submitted to an insurance provider for reimbursement. The claim data is categorized to group together claim data from a same person. Illustrated processing block 454 trains a first machine learning model based on the claim data to identify treatments a user will receive based on the claim data. Illustrated processing block 456 trains a second machine learning model based on the claim data and outputs of the first machine learning model to identify timings of the treatment. The claims data can accurately disclose the treatments that are executed for various diseases, as well as timings. Accordingly, the claims data can provide an accurate historical record of treatments, as well as outcomes (e.g., timeline for treatments, whether a treatment was unsuccessful or successful, etc.).

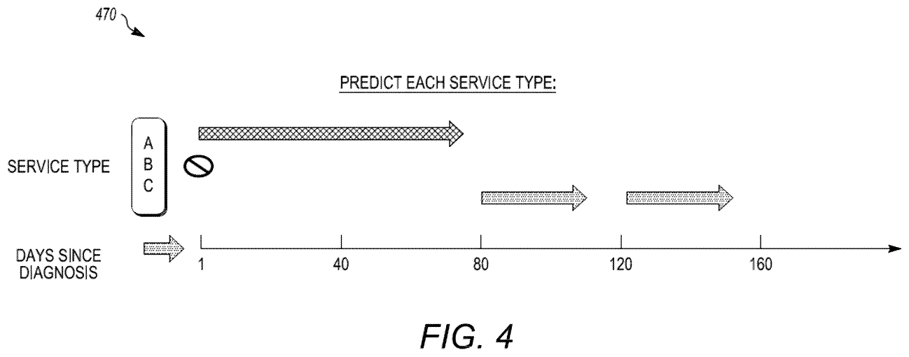

FIG. 4 illustrates a modeling medical visit sequences 470. The modeling medical visit sequences 470 can be readily implemented in the example of the enhanced prediction process 120 (FIG. 1), method 400 (FIG. 2) and/or method 450 (FIG. 3).

The modeling medical visit sequences 470 uses a sequence of model pairs such as the first ML model 122 and the second ML model 130 (FIG. 1). The model pair includes a first model to predict if a medical visit will occur, and a second model to predict when the medical visit will occur, given that the medical visit will occur. Each model pair is trained at fixed points in time from “date of diagnosis” (e.g., 21 points in time (8-days apart); for 5 service types, 210 models). In this example a service type includes A, B or C. Days since diagnosis is also shown along the horizontal axis. Initially, a service type of A is predicted to occur at around 80 days. Following the service type of A, a service type of C is predicted to occur at around 90 days from diagnosis, and again at 150 days from the diagnosis.

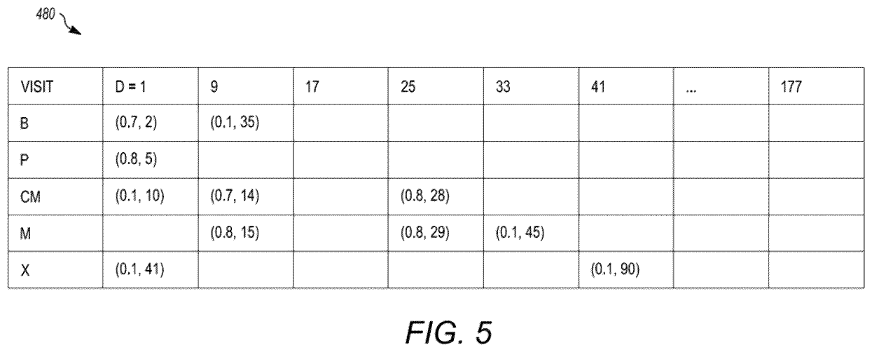

FIG. 5 illustrates a tabular output 480 according to embodiments as described herein. The tabular output 480 can be readily implemented in the example of the enhanced prediction process 120 (FIG. 1), method 400 (FIG. 2), method 450 (FIG. 3) and/or modeling medical visit sequences 470 (FIG. 4). Consider various treatment: V_{d}=[P,M,E,R,B,S,T,X,Cr,Cm], where P is Pathology, M is Medication, E is durable medical equipment (DME) and Supplies, R is Radiology, B is Biopsy, S is Surgery, T is Therapeutic, X is therapeutic radiation; Cr is Chemotherapy (Rx) and Cm is Chemotherapy. As noted, the d=8-day intervals and examples predict two things including what and when a service will happen. As illustrated, various probabilities for each visit and treatment are provided. For example, the 0.7 would change B and it will happens on day 2.

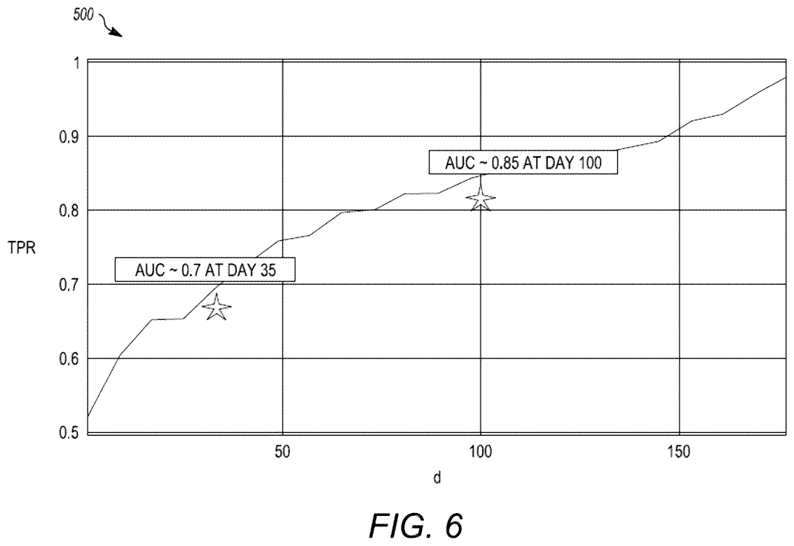

FIG. 6 illustrates a probability of treatment graph 500 mapped to days. The probability of treatment graph 500 can be readily implemented in enhanced prediction process 120 (FIG. 1), method 400 (FIG. 2), method 450 (FIG. 3), modeling medical visit sequences 470 (FIG. 4) and/or tabular output 480 (FIG. 5).

Enhanced examples described herein generate the probability of treatment graph 500. The enhanced examples predict no future treatment will occur. That is, the enhanced examples accurately predict that no visit will occur by day 180. A round 35 days after Dx (diagnosis shown at day 0) there is a 0.70 true positive rate (TPR) for correctly predicting no event will occur, and around 100 days after diagnoses examples have 0.85 TPR for correctly predicting no event will occur. Accordingly, examples herein can identify those users that are unlikely to need coaching or other interventions and adjust automated systems accordingly to bypass emails, text messages, etc. to such users. Some of this strength is due to detecting the hallmarks of a sequence of care ending.

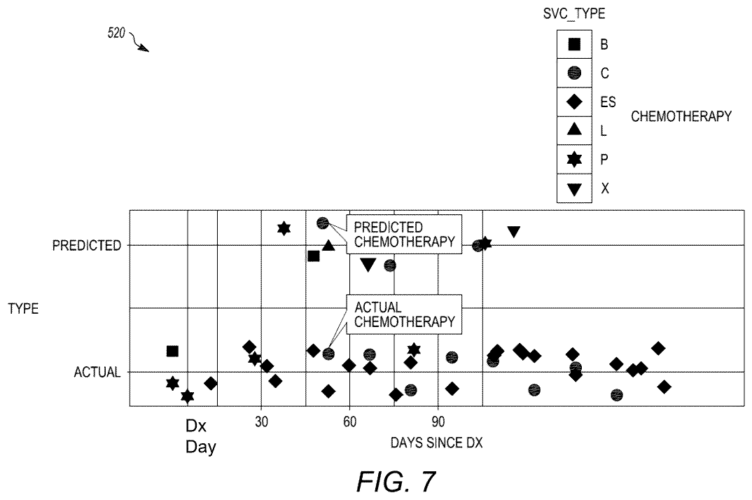

FIG. 7 illustrates a graph 520 predicted chemotherapy treatments as predicted by enhanced examples herein compared to actual treatments. The graph 520 can be readily implemented in conjunction with and/or as part of enhanced prediction process 120 (FIG. 1), method 400 (FIG. 2), method 450 (FIG. 3), modeling medical visit sequences 470 (FIG. 4), tabular output 480 (FIG. 5) and/or probability of treatment graph 500 (FIG. 6).

The treatments shown in graph 520 can relate to breast cancer and predict a true cancer five days from a first visit. As illustrated, the predicted chemotherapy aligns closely with the actual chemotherapy.

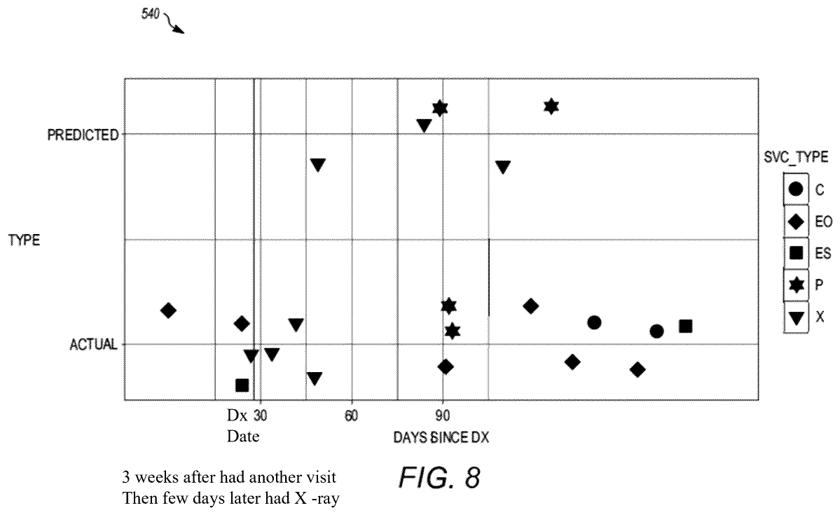

FIG. 8 illustrates a breast cancer prediction graph 540 that is generated by enhanced examples herein. The breast cancer prediction graph 540 can be readily implemented in conjunction with and/or as part of enhanced prediction process 120 (FIG. 1), method 400 (FIG. 2), method 450 (FIG. 3), modeling medical visit sequences 470 (FIG. 4), tabular output 480 (FIG. 5), probability of treatment graph 500 (FIG. 6) and/or graph 520 (FIG. 7).

The prediction in breast cancer prediction graph 540 is not a cancer thirty days after a medical visit. From clinical perspective, the symptoms exhibited by a corresponding patient are insignificant, so no immediate follow up visits or scans are suggested.

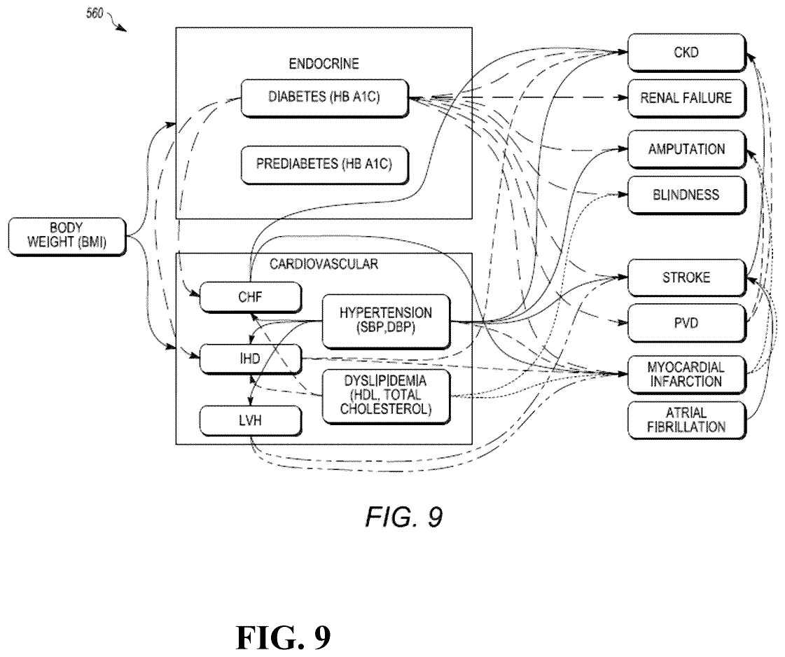

FIG. 9 illustrates a Cardiometabolic Syndrome (CM S) 560 (e.g., disease). CM S is a group of common but often preventable conditions that affect cardiovascular systems and metabolic health. The flow is illustrated to show how health data can be used to lead to inferences of increased risk of CM S 560 and can be learned with the first ML model 122 and the second ML model 130 (FIG. 1).

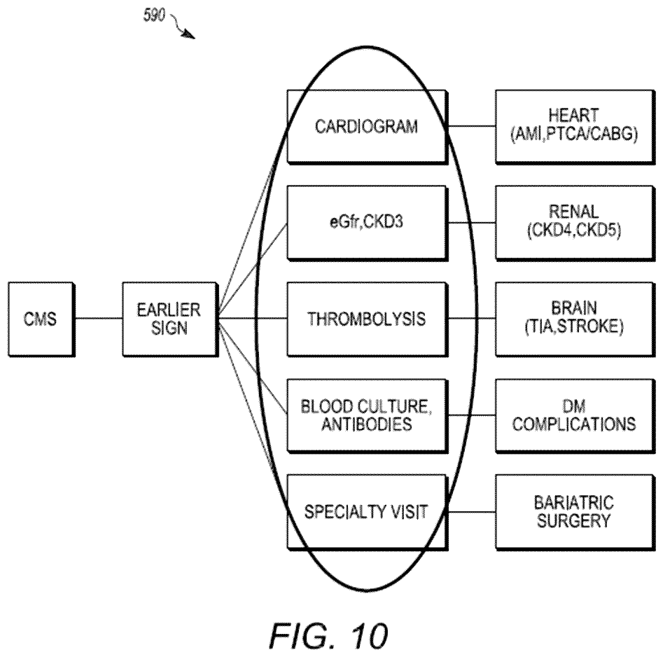

FIG. 10 illustrates a key indicators 590 that lead to specific pathway. A machine learning model, such as the first ML model 122 and the second ML model 130 (FIG. 1), as described herein can learn such patterns and key indicators 590 to generate inferences about treatments and timings of such treatments.

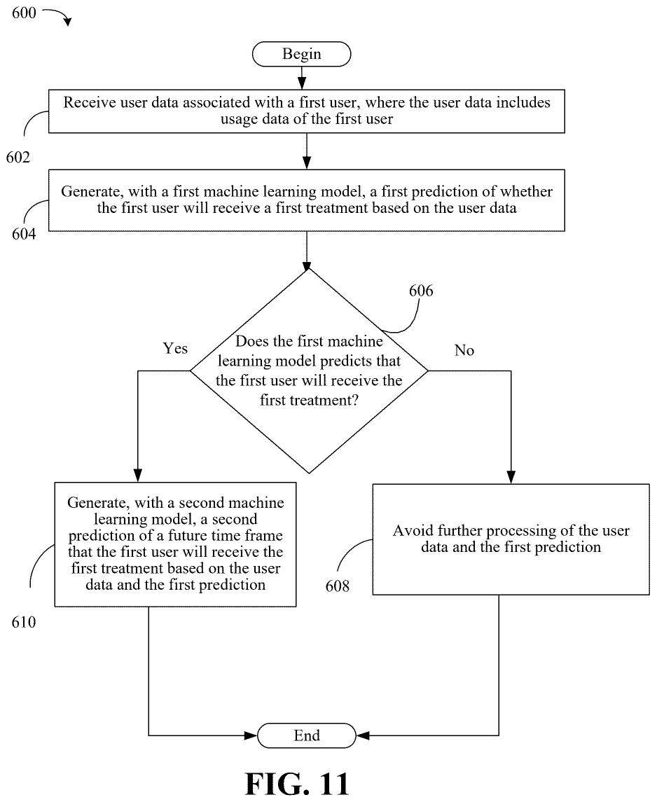

FIG. 11 illustrates a method 600 that remediates missing data. The method 600 can generally be implemented in conjunction with any of the embodiments described herein, for example the enhanced prediction process 120 (FIG. 1), method 400 (FIG. 2), method 450 (FIG. 3), modeling medical visit sequences 470 (FIG. 4), tabular output 480 (FIG. 5), probability of treatment graph 500 (FIG. 6), graph 520 (FIG. 7), breast cancer prediction graph 540 (FIG. 8), CMS 560 (FIG. 9) and/or key indicators 590 (FIG. 10). In an embodiment, the method 600 is implemented in logic instructions (e.g., software), configurable logic, fixed-functionality hardware logic, computer readable instructions stored on at least one non-transitory computer readable storage medium that are executable to implement method 600, circuitry, etc., or any combination thereof.

Illustrated processing block 602 receives user data associated with a first user, where the user data includes usage data of the first user. Illustrated processing block 604 generates, with a first machine learning model, a first prediction of whether the first user will receive a first treatment based on the user data. Illustrated processing block 606 determines if the first machine learning model predicts that the first user will receive the first treatment. If so (if the first machine learning model predicts that the first user will receive the first treatment), illustrated processing block 610 generates, with a second machine learning model, a second prediction of a future time frame that the first user will receive the first treatment based on the user data and the first prediction. Otherwise (if the first machine learning model does not predict that the first user will receive the first treatment), illustrated processing block 608 avoids further processing of the user data and the first prediction.

In some examples, the method 600 includes determining, with the first machine learning model, a plurality of predictions based on the user data, where the plurality of predictions include different treatments of the first user that are associated with different probabilities and conditions of the first user, grouping the plurality of predictions into different groups, and selecting the first prediction from the plurality of predictions based on the first prediction having a probability from the different probabilities that is above a threshold and being grouped into a high-risk group from the different groups. In some examples, the first prediction includes a second treatment that follows the first treatment, and the second prediction includes a second future time frame that the first user will receive the second treatment.

In some examples, the method 600 includes determining a previous treatment associated with the first user, and identifying the first treatment based on the previous treatment. In some examples, the first machine learning model is a single model, and the second machine learning model is a single model. The method 600 can further identify, with the second machine learning model, a date that the first user is identified as having a condition, and determine the future time frame based on the date, where the future time frame is a number of days from the date. The user data includes claim records of the first user.

In some examples, processing block 604 includes categorizing, with the first machine learning model, the first user into a first group from a plurality of groups based on the user data, and generating the first prediction based on the first group. In some examples, the method 600 includes identifying total data associated with the first user, determining a window of time based on a history of the first user, and filtering the total data based on the window of time to generate the user data.

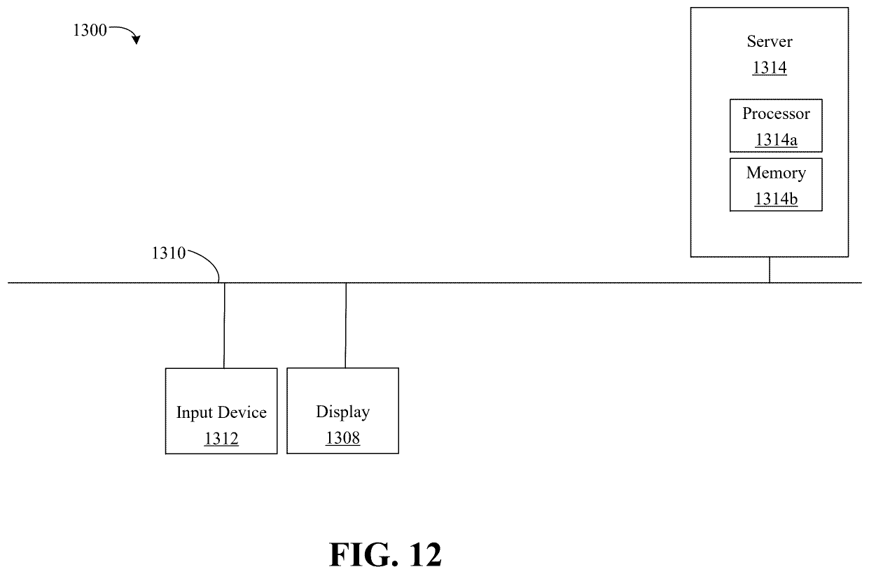

FIG. 12 shows a more detailed example of a computing architecture 1300 to execute a digital prediction and timing process. The computing architecture 1300 can be implemented in conjunction with and/or by any of the examples described herein, such as the enhanced prediction process 120 (FIG. 1), method 400 (FIG. 2), method 450 (FIG. 3), modeling medical visit sequences 470 (FIG. 4), tabular output 480 (FIG. 5), probability of treatment graph 500 (FIG. 6), graph 520 (FIG. 7), breast cancer prediction graph 540 (FIG. 8), CMS 560 (FIG. 9), key indicators 590 (FIG. 10) and/or method 600 (FIG. 11).

In the illustrated example, the computing architecture 1300 can include a network 1310 that can facilitate communication between server 1314, input device 1312, and display 1308. The display 1308 (e.g., audio and/or visual interface) can present treatment sequences and timings to a user that is to receive treatments of the treatment sequences, and the input device 1312 can receive user inputs (e.g., select particular treatments). In some examples, the user can manipulate a graphical user interface through the input device 1312 to select one or more of the treatments of the treatment sequences to obtain more information and/or different providers to execute the treatments.

The server 1314 includes a processor 1314a (e.g., embedded controller, central processing unit/CPU) and a memory 1314b (e.g., non-volatile memory/NVM and/or volatile memory) containing a set of instructions, which when executed by the processor 1314a, cause the server 1314 to implement aspects described herein such as enhanced prediction process 120 (FIG. 1) for example.

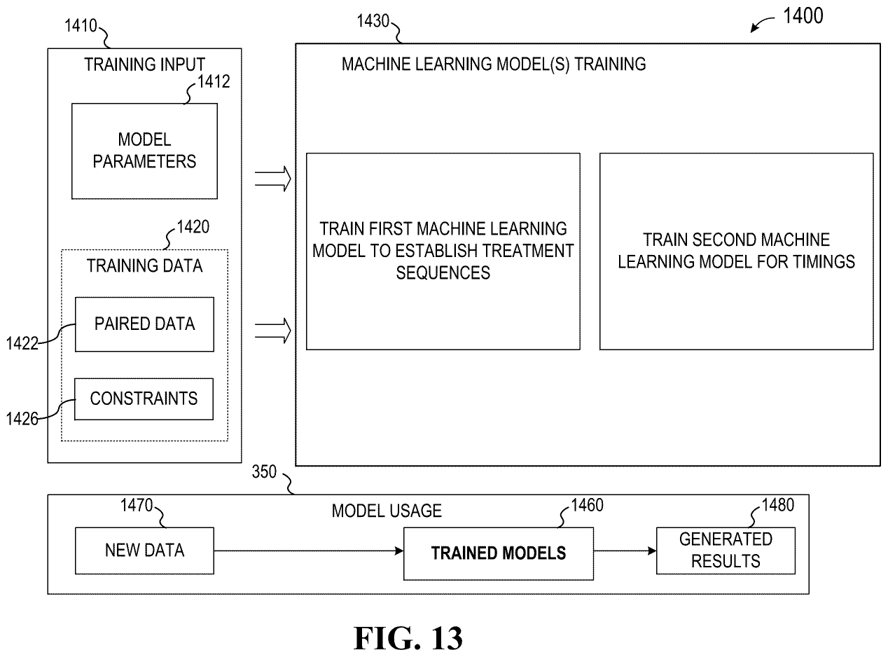

FIG. 13 is a block diagram of an example service of enhanced automated treatment prediction and timing/system 1400 that may be deployed within and/or in conjunction with the examples herein, including enhanced prediction process 120 (FIG. 1). Training input 1410 includes model parameters 1412 and training data 1420, which may include paired training data sets 1422 (e.g., input-output training pairs including diagnoses, treatments, and timings) and constraints 1426. Model parameters 1412 store or provide the parameters or coefficients of corresponding ones of machine learning models. During training, these parameters 1412 are adapted based on the input-output training pairs of the training data sets 1422. After the parameters 1412 are adapted (after training), the parameters are used by trained models 1460 to implement the trained machine learning models on a new set of data 1470 from model usage 350.

Training data 1420 includes constraints 1426 which may define the constraints of a given patient information features. The paired training data sets 1422 may include sets of input-output pairs, such as pairs of a plurality of training medical data (e.g., insurance claims, electronic health records, etc.) and features of post patient analysis that are created in association with one or more of the training medical data (e.g., ground-truth successful patient treatments and timings for a diagnosis, and unsuccessful patient treatments and timings for the diagnosis). Some components of training input 1410 may be stored separately at a different off-site facility or facilities than other components.

Machine learning model(s) training 1430 trains one or more machine learning techniques based on the sets of input-output pairs of paired training data sets 1422. For example, the model training 1430 may train the machine learning (ML) model parameters 1412 by minimizing a loss function based on one or more ground-truth successful patient treatments. The ML model can include any one or combination of classifiers or neural networks, such as an artificial neural network, a convolutional neural network, an adversarial network, a generative adversarial network, a deep feed forward network, a radial basis network, a recurrent neural network, a long/short term memory network, a gated recurrent unit, an auto encoder, a variational autoencoder, a denoising autoencoder, a sparse autoencoder, a Markov chain, a Hopfield network, a Boltzmann machine, a restricted Boltzmann machine, a deep belief network, a deep convolutional network, a deconvolutional network, a deep convolutional inverse graphics network, a liquid state machine, an extreme learning machine, an echo state network, a deep residual network, a Kohonen network, a support vector machine, a neural Turing machine, an LLM, a generative network, a diffusion model, and the like.

Particularly, the ML model can be applied to a training batch of medical features to estimate or generate one or more preliminary patient predictions and times. In some implementations, a derivative of a loss function is computed based on a comparison of the preliminary patient digital predictions and times and the ground truth successful patient treatments and timings for a diagnosis, and parameters of the ML model are updated based on the computed derivative of the loss function.

The result of minimizing the loss function for multiple sets of training data trains, adapts, or optimizes the model parameters 1412 of the corresponding ML models. In this way, the ML model is trained to establish a relationship between medical data and ground-truth patient treatments and timings.

After the machine learning model is trained, new data 1470, including medical data can be provided. The trained machine learning model may be applied to the new data 1470 to generate results 1480 including timings and treatments.

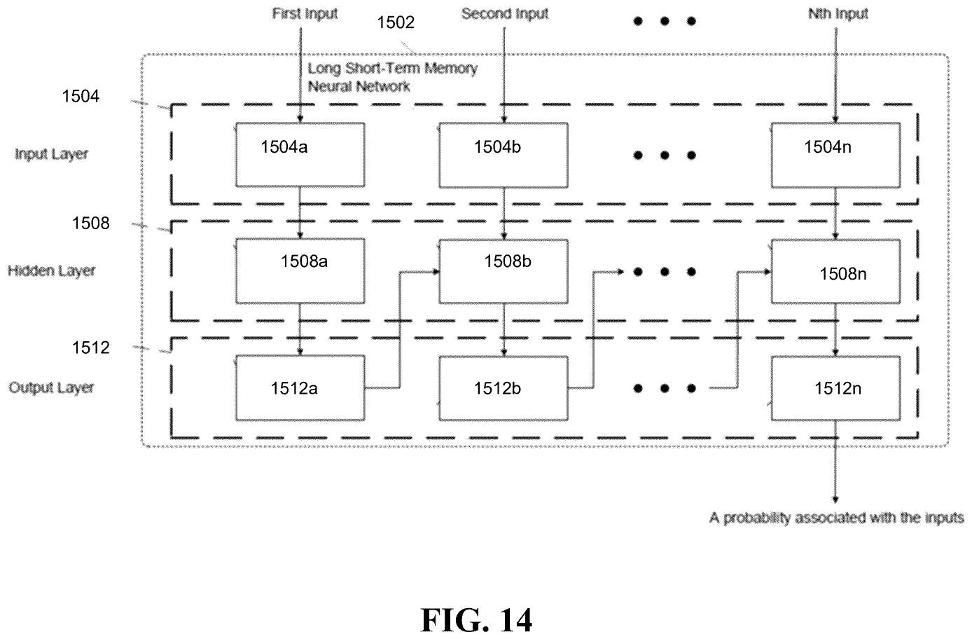

FIG. 14 is a functional block diagram of an example neural network 1502 that can be used for the inference engine or other functions (e.g., engines) as described herein to produce a predictive model. Neural network 1502 may be deployed within and/or in conjunction with the examples herein, including the enhanced prediction process 120 (FIG. 1), method 400 (FIG. 2), method 450 (FIG. 3), modeling medical visit sequences 470 (FIG. 4), tabular output 480 (FIG. 5), probability of treatment graph 500 (FIG. 6), graph 520 (FIG. 7), breast cancer prediction graph 540 (FIG. 8), CMS 560 (FIG. 9), key indicators 590 (FIG. 10) and/or method 600 (FIG. 11) according to some examples. In particular, the example neural network 1502 can be readily substitute for the first ML model 122 and the second ML model 130. The predictive model can generate treatments and corresponding timings. In an example, the neural network 1502 can be a LSTM neural network. In an example, the neural network 1502 can be a recurrent neural network (RNN). The example neural network 1502 may be used to implement the machine learning as described herein, and various implementations may use other types of machine learning networks. The neural network 1502 includes an input layer 1504, a hidden layer 1508, and an output layer 1512. The input layer 1504 includes inputs 1504a, 1504b . . . 1504n. The hidden layer 1508 includes neurons 1508a, 1508b . . . 1508n. The output layer 1512 includes outputs 1512a, 1512b . . . 1512n.

Each neuron of the hidden layer 1508 receives an input from the input layer 1504 and outputs a value to the corresponding output in the output layer 1512. For example, the neuron 1508a receives an input from the input 1504a and outputs a value to the output 1512a. Each neuron, other than the neuron 1508a, also receives an output of a previous neuron as an input. For example, the neuron 1508b receives inputs from the input 1504b and the output 1512a. In this way the output of each neuron is fed forward to the next neuron in the hidden layer 1508. The last output 1512n in the output layer 1512 outputs a probability associated with the inputs 1504a-1504n. Although the input layer 1504, the hidden layer 1508, and the output layer 1512 are depicted as each including three elements, each layer may contain any number of elements. Neurons can include one or more adjustable parameters, weights, rules, criteria, or the like.

In various implementations, each layer of the neural network 1502 must include the same number of elements as each of the other layers of the neural network 1502. For example, training can include generating timings and treatments that are compared to ground-truths, and may be processed to create the inputs 1504a-1504n. The neural network 1502 may implement a model to produce one or more preliminary predictions and timings. More specifically, the inputs 1504a-1504n can include fields of the user features as data features (binary, vectors, factors or the like) stored in the storage device. The fields of the user features as data features can be provided to neurons 1508a-1508n for analysis and connections between the known facts. The neurons 1508a-1508n, upon finding connections, provides the potential connections as outputs to the output layer 1512, which determines a preliminary post patient encounter document.

The neural network 1502 can perform any of the above calculations. The output of the neural network 1502 can be used to determine treatments, timings, as well as whether and when to contact a user digitally.

In some examples, a convolutional neural network may be implemented. Similar to neural networks, convolutional neural networks include an input layer, a hidden layer, and an output layer. However, in a convolutional neural network, the output layer includes one fewer output than the number of neurons in the hidden layer and each neuron is connected to each output. Additionally, each input in the input layer is connected to each neuron in the hidden layer. In other words, input 1504a is connected to each of neurons 1508a, 1508b . . . 1508n.

The present systems and methods can extract and process medical data from medical insurance claims to assist in determining whether to perform a treatment and corresponding timing thereof. Times and/or the probability can be determined using the neural network (generative AI engine) associated with the conversation. A predictive-timing neural network can receive medical data, or combinations thereof to determine a timing and treatments of the patient. The present system can use systems and methods from U.S. Pat. No. 11,031,013, which is assigned to the present assignee and incorporated herein by reference.

“COMPONENT” in this context refers to a device, physical entity, or logic having boundaries defined by function or subroutine calls, branch points, APIs, or other technologies that provide for the partitioning or modularization of particular processing or control functions. Components may be combined via their interfaces with other components to carry out a machine process. A component may be a packaged functional hardware unit designed for use with other components and a part of a program that usually performs a particular function of related functions. Components may constitute either software components (e.g., code embodied on a machine-readable medium) or hardware components. A “hardware component” is a tangible unit capable of performing certain operations and may be configured or arranged in a certain physical manner. In various example embodiments, one or more computer systems (e.g., a standalone computer system, a client computer system, or a server computer system) or one or more hardware components of a computer system (e.g., a processor or a group of processors) may be configured by software (e.g., an application or application portion) as a hardware component that operates to perform certain operations as described herein.

A hardware component may also be implemented mechanically, electronically, or any suitable combination thereof. For example, a hardware component may include dedicated circuitry or logic that is permanently configured to perform certain operations. A hardware component may be a special-purpose processor, such as a Field-Programmable Gate Array (FPGA) or an A SIC. A hardware component may also include programmable logic or circuitry that is temporarily configured by software to perform certain operations. For example, a hardware component may include software executed by a general-purpose processor or other programmable processor. Once configured by such software, hardware components become specific machines (or specific components of a machine) uniquely tailored to perform the configured functions and are no longer general-purpose processors. It will be appreciated that the decision to implement a hardware component mechanically, in dedicated and permanently configured circuitry, or in temporarily configured circuitry (e.g., configured by software) may be driven by cost and time considerations. Accordingly, the phrase “hardware component”(or “hardware-implemented component”) should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware components are temporarily configured (e.g., programmed), each of the hardware components need not be configured or instantiated at any one instance in time. For example, where a hardware component comprises a general-purpose processor configured by software to become a special-purpose processor, the general-purpose processor may be configured as respectively different special-purpose processors (e.g., comprising different hardware components) at different times. Software accordingly configures a particular processor or processors, for example, to constitute a particular hardware component at one instance of time and to constitute a different hardware component at a different instance of time.

Hardware components can provide information to, and receive information from, other hardware components. Accordingly, the described hardware components may be regarded as being communicatively coupled. Where multiple hardware components exist contemporaneously, communications may be achieved through signal transmission (e.g., over appropriate circuits and buses) between or among two or more of the hardware components. In embodiments in which multiple hardware components are configured or instantiated at different times, communications between such hardware components may be achieved, for example, through the storage and retrieval of information in memory structures to which the multiple hardware components have access. For example, one hardware component may perform an operation and store the output of that operation in a memory device to which it is communicatively coupled. A further hardware component may then, at a later time, access the memory device to retrieve and process the stored output.

Hardware components may also initiate communications with input or output devices (e.g., from the automated communication system to the patient devices or from the stacked ensemble engine to the automated communication system) and can operate on a resource (e.g., a collection of information). The various operations of example methods described herein may be performed, at least partially, by one or more processors that are temporarily configured (e.g., by software) or permanently configured to perform the relevant operations. Whether temporarily or permanently configured, such processors may constitute processor-implemented components that operate to perform one or more operations or functions described herein. As used herein, “processor-implemented component” refers to a hardware component implemented using one or more processors. Similarly, the methods described herein may be at least partially processor-implemented, with a particular processor or processors being an example of hardware. For example, at least some of the operations of a method may be performed by one or more processors or processor-implemented components. Moreover, the one or more processors may also operate to support performance of the relevant operations in a “cloud computing” environment or as a “software as a service” (Saas). For example, at least some of the operations may be performed by a group of computers (as examples of machines including processors), with these operations being accessible via a network (e.g., the Internet) and via one or more appropriate interfaces (e.g., an API). The performance of certain of the operations may be distributed among the processors, not only residing within a single machine, but deployed across a number of machines. In some example embodiments, the processors or processor-implemented components may be located in a single geographic location (e.g., within a home environment, an office environment, or a server farm). In other example embodiments, the processors or processor-implemented components may be distributed across a number of geographic locations.

The term “coupled” can be used herein to refer to any type of relationship, direct or indirect, between the components in question, and can apply to electrical, mechanical, fluid, optical, electromagnetic, electromechanical or other connections. In addition, the terms “first”, “second”, etc. can be used herein only to facilitate discussion, and carry no particular temporal or chronological significance unless otherwise indicated.

Those skilled in the art will appreciate from the foregoing description that the broad techniques of the embodiments of the present disclosure can be implemented in a variety of forms. Therefore, while the embodiments of this disclosure have been described in connection with particular examples thereof, the true scope of the embodiments of the disclosure should not be so limited since other modifications will become apparent to the skilled practitioner upon a study of the drawings, specification, and following claims.

1. A computing system comprising:
a processor; and
a memory having a set of instructions, which when executed by the processor, cause the computing system to:
receive first user data associated with a first user, wherein the first user data includes usage data of the first user;
generate, with a first machine learning model, a first prediction of whether the first user will receive a first plurality of treatments based on the first user data, wherein the first plurality of treatments is different from each other, wherein the first machine learning model is a single model;
in response to the first machine learning model predicting that the first user will receive the first plurality of treatments, provide the first plurality of treatments to a second machine learning model, and generate, with the second machine learning model, a second prediction of a plurality of treatment-specific future time frames indicating, for each respective treatment in the first plurality of treatments, a respective treatment-specific future time frame at which the first user will receive the respective treatment;
determine, with the first machine learning model, a third prediction of whether a second user will receive a second treatment based on second user data; and
filter the third prediction by selectively providing the third prediction and the second user data to the second machine learning model only when the third prediction indicates that the second user will receive the second treatment.

2. The computing system of claim 1, wherein the instructions of the memory, when executed, cause the computing system to:
determine different probabilities for conditions of the first user associated with the first plurality of treatments;
group the the conditions into different groups; and
select a selected treatment from the first plurality of treatments based on the selected treatment being associated with a condition of the conditions having a probability from the different probabilities that is above a threshold and being grouped into a high-risk group from the different groups.

3. The computing system of claim 1, wherein the first plurality of treatments includes a following treatment that follows an initial treatment of the first plurality of treatments.

4. The computing system of claim 1, wherein the instructions of the memory, when executed, cause the computing system to:
determine a previous treatment associated with the first user; and
identify the first plurality of treatments based on the previous treatment.

5. The computing system of claim 1, wherein the second machine learning model is a single model.
wherein the instructions of the memory, when executed, cause the computing system to execute electronic outreach based on the plurality of treatment-specific future time frames.

6. The computing system of claim 1, wherein the instructions of the memory, when executed, cause the computing system to:
identify, with the second machine learning model, a date that the first user is identified as having a condition; and
determine the plurality of treatment-specific future time frames based on the date, wherein each of the plurality of treatment-specific future time frames is a different number of days from the date.

7. The computing system of claim 1, wherein the first user data includes claim records of the first user.

8. The computing system of claim 1, wherein to generate the first prediction, the instructions of the memory, when executed, cause the computing system to:
categorize, with the first machine learning model, the first user into a first group from a plurality of groups based on the first user data; and
generate the first prediction based on the first group.

9. The computing system of claim 1, wherein the instructions of the memory, when executed, cause the computing system to:
identify total data associated with the first user;
determine a window of time based on a history of the first user; and
filter the total data based on the window of time to generate the first user data.

10. At least one non-transitory computer readable storage medium comprising a set of instructions, which when executed by a computing system, cause the computing system to:
receive first user data associated with a first user, wherein the first user data includes usage data of the first user;
generate, with a first machine learning model, a first prediction of whether the first user will receive a first plurality of treatments based on the first user data, wherein the first plurality of treatments is different from each other, wherein the first machine learning model is a single model;
in response to the first machine learning model predicting that the first user will receive the first plurality of treatments, provide the first plurality of treatments to a second machine learning model, and generate, with the second machine learning model, a second prediction of a plurality of treatment-specific future time frames indicating, for each respective treatment in the first plurality of treatments, a respective treatment-specific future time frame at which the first user will receive the respective treatment;
determine, with the first machine learning model, a third prediction of whether a second user will receive a second treatment based on second user data; and
filter the third prediction by selectively providing the third prediction and the second user data to the second machine learning model only when the third prediction indicates that the second user will receive the second treatment.

11. The at least one non-transitory computer readable storage medium of claim 10, wherein the instructions, when executed, cause the computing system to:
determine different probabilities for conditions of the first user associated with the first plurality of treatments;
group the conditions into different groups; and
select a selected treatment from the first plurality of treatments based on the selected treatment being associated with a condition of the conditions having a probability from the different probabilities that is above a threshold and being grouped into a high-risk group from the different groups.

12. The at least one non-transitory computer readable storage medium of claim 10, wherein the first plurality of treatments includes a following treatment that follows an initial treatment of the first plurality of treatments.

13. The at least one non-transitory computer readable storage medium of claim 10, wherein the instructions, when executed, cause the computing system to:
determine a previous treatment associated with the first user; and
identify the first plurality of treatments based on the previous treatment.

14. The at least one non-transitory computer readable storage medium of claim 10, wherein the second machine learning model is a single model,
wherein the instructions, when executed, cause the computing system to execute electronic outreach based on the plurality of treatment-specific future time frames.

15. The at least one non-transitory computer readable storage medium of claim 10, wherein the instructions, when executed, cause the computing system to:
identify, with the second machine learning model, a date that the first user is identified as having a condition; and
determine the plurality of treatment-specific future time frames based on the date, wherein each of the plurality of treatment-specific future time frames is a different number of days from the date.

16. The at least one non-transitory computer readable storage medium of claim 10, wherein the first user data includes claim records of the first user.

17. The at least one non-transitory computer readable storage medium of claim 10, wherein the instructions, when executed, cause the computing system to:
categorize, with the first machine learning model, the first user into a first group from a plurality of groups based on the first user data; and
generate the first prediction based on the first group.

18. The at least one non-transitory computer readable storage medium of claim 10, wherein the instructions, when executed, cause the computing system to:
identify total data associated with the first user;
determine a window of time based on a history of the first user; and
filter the total data based on the window of time to generate the first user data.

19. A method executed by a computing system, the method comprising:
receiving first user data associated with a first user, wherein the first user data includes usage data of the first user;
generate, with a first machine learning model executed by the computing system, a first prediction of whether the first user will receive a first plurality of treatments based on the first user data, wherein the first plurality of treatments are different from each other, wherein the first machine learning model is a single model;
in response to the first machine learning model predicting that the first user will receive the first plurality of treatments, provide the first plurality of treatments to a second machine learning model of the computing system, and generate, with the second machine learning model, a second prediction of a plurality of treatment-specific future time frames indicating, for each respective treatment in the first plurality of treatments, a respective treatment-specific future time frame at which the first user will receive the respective treatment;
determine, with the first machine learning model, a third prediction of whether a second user will receive a second treatment based on second user data; and
filter the third prediction by selectively providing the third prediction and the second user data to the second machine learning model only when the third prediction indicates that the second user will receive the second treatment.

20. The method of claim 19, wherein the second machine learning model is a single model.
the method further comprising executing electronic outreach based on the plurality of treatment-specific future time frames.
